(19) United States
(12) Patent Application Publication
(10) Pub. No.: US 2018/0015066 A1
(43) Pub. Date: Jan. 18, 2018

(54) DRUG COMPLEXES COMPRISING ALPHA-FETOPROTEIN

(71) Applicant: UNIVERSITY HEALTH NETWORK, Toronto (CA)

(72) Inventors: Lakshmi P. Kotra, Thornhill (CA); Christopher J. Paige, Toronto (CA); Angelica M. Bello, Toronto (CA); Igor Sherman, Toronto (CA)

(21) Appl. No.: 15/546,937

(22) PCT Filed: Jan. 26, 2016
(86) PCT No.: PCT/CA2016/000023

§ 371 Date: Jul. 27, 2017

Related U.S. Application Data
(60) Provisional application No. 62/108,609, filed on Jan. 28, 2015.

(51) Int. Cl. A61K 31/337; A61K 47/42; A61K 9/00; A61K 9/19; C07K 14/47; C07D 305/14

(52) U.S. Cl. CPC A61K 31/337; C07K 14/4715; C07D 305/14; A61K 9/0019; A61K 9/19; A61K 47/42

(57) ABSTRACT

Compositions which comprise a hydro-phobic taxane such as paclitaxel are produced by non-covalent complexing between the taxane and alpha-fetoprotein c at a ratio of about 4 moles of taxane per mole of AFP. The complexes are water soluble and suitable for injection. Uses of the compositions for treating a subject presenting with an AFP receptor positive and taxane responsive ease cell are also disclosed.

### FIELD OF THE INVENTION

This invention relates to drug complexes and formulations thereof that are useful in the treatment of cancer and other diseases and conditions. The invention relates particularly to formulations comprising a taxane such as paclitaxel.

### BACKGROUND OF THE INVENTION

Paclitaxel is a secondary metabolite that is extractable from the bark of the Pacific yew tree and used in the treatment of various cancers including head and neck, breast and ovarian cancers. It is also useful to promote revascularization, in the treatment of restenosis, and in the treatment of non-small cell lung cancer and AIDS-related Kaposi's sarcoma. It acts by arresting the microtubules of cells, thereby preventing normal cell division and causing a G2/M phase blockage. Despite its complex chemical structure, shown below, total synthetic production has been achieved:

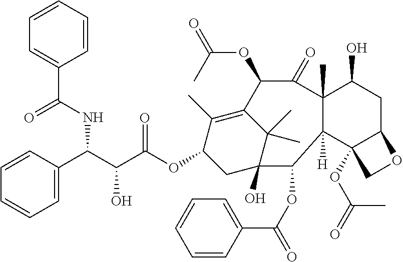 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

Paclitaxel is formulated for administration by infusion or by injection, yet is poorly soluble in water. Current paclitaxel formulations therefore use either non-aqueous solvents, such as DMSO, or they incorporate water-miscible solubilizers or lipid-based emulsions. In a marketed formulation, paclitaxel is mixed with Cremophor-EL (polyethoxylated castor oil) and ethanol, which transforms spontaneously into a microemulsion when diluted in sterile saline for administration. This vehicle itself is associated with severe, life-threatening hypersensitivity reactions. There have accordingly been numerous efforts to improve upon the formulation of paclitaxel.

More recently, paclitaxel has been formulated with aggregated human albumin, to form non-covalent nanoparticulate complexes that are then formulated for administration and sold under the name Abraxane®. This product shows reduced toxicity relative to the Cremophor-based formulation at equal doses. Microparticulate liposome-based paclitaxel formulations have also been described. As well, particulate paclitaxel formulations consisting of a tocopherol nanoemulsion have progressed to clinical trials, although with evidence that unbound paclitaxel can be released from the formulation to create pharmacokinetic and toxicity concerns.

The need to reduce paclitaxel toxicity and thereby reduce adverse clinical events and to improve efficacy has led to the development of paclitaxel conjugates, in which paclitaxel is chemically linked to a carrier molecule that binds selectively to an endogenous tumour target, and thereby reduces normal tissue exposure to paclitaxel. In this approach, paclitaxel is chemically conjugated to the carrier, usually an antibody, using a chemical linker that is labile to conditions present in the target tumour cell. Thus, the administered conjugate binds via the antibody to an antigen unique to the tumour cell being treated, the conjugate is taken up by the cell, and the paclitaxel is then released intracellularly to exert its effect selectively within the target cell. The presence of the conjugated antibody can insulate the recipient from the toxic effects of the administered paclitaxel on healthy tissues and allow greater amounts of the drug to reach cancer cells. Formulating antibody-conjugated paclitaxel can also be approached using vehicles that are otherwise standard in the art of protein formulation, and thereby reduce or eliminate the need for solubility enhancers that can be toxic in themselves. It will be appreciated, however, that the production of paclitaxel as an antibody conjugate contributes significantly to the cost of the drug, and can alter its pharmacokinetics and efficacy.

The challenges faced when formulating paclitaxel are also encountered when formulating structurally related compounds that are also useful in the treatment of diseases including cancer. Particularly, paclitaxel is a member of the taxane family characterized by a diterpene structure. This family includes the drug docetaxel, which is marketed for cancer treatment under the proprietary name Taxotere®. The family also includes salts and esters of these two taxanes, including paclitaxel succinate, for instance. Although docetaxel is somewhat more water soluble than paclitaxel, it too requires formulation with organic solvents such as ethanol and anhydrous polysorbate.

There remains a continuing need for alternative formulations, including alternative paclitaxel formulations.

### SUMMARY OF THE INVENTION

There is now provided a pharmaceutical formulation that provides paclitaxel or a related taxane in a form that is soluble in aqueous vehicles such as saline or water. The taxane furthermore is provided in a controlled unit dosage form that permits ready calculation of administered doses. Moreover, the taxane is provided in a form that enables it to be delivered selectively to certain cancer cells, thus sparing healthy, normal cells from taxane-induced damage.

According to one aspect of the present invention, there is provided a complex comprising a taxane and α-fetoprotein (AFP), wherein the AFP and taxane are complexed non-covalently and the taxane is present in the complex at about 4 molecules per molecule of AFP. In embodiments, the taxane is present at about 3 to about 5 molecules per molecule of AFP. In a related aspect, the complex is provided as a preparation that is essentially free from taxane in a non-complexed state. The preparation can be provided as a lyophilized preparation, for reconstitution in aqueous vehicle and subsequent administration to a subject in need thereof.

The AFP complexed with the taxane effectively presents the complexed taxane selectively to cells that are positive for the AFP receptor, the vast majority of such cells being disease cells including particularly cancer cells. The complex thus provides a means for delivering the toxic taxane, as payload, selectively to disease cells thereby sparing normal cells and tissues from the adverse effects of the taxane. As well, formation of the complex can be controlled in such a way that non-complexed taxane is absent, and only complexed taxane is then formulated for administration. Control over the complex formation also allows for production of complexes that incorporate a known ratio of taxane to AFP, so that taxane formulation and dosing can be calculated and controlled.

In other aspects, the present invention provides a pharmaceutical composition comprising a pharmaceutically acceptable carrier and a taxane:AFP complex of the present invention.

In related aspects of the present invention, there is provided a method for inhibiting the growth or proliferation of a disease cell that is AFP receptor positive and taxane-responsive, comprising treating the cell with an effective amount of complex of the present invention. As well, in another aspect, the present invention provides a method for treating a subject presenting with an AFP receptor positive disease cell, comprising administering to the subject an amount of the present complex effective to inhibit the growth or proliferation of the disease cell. In embodiments, the effective amount of taxane in the administered complex is an amount lower than would be required for the same effect with free taxane.

In a further aspect, the present invention provides a method for obtaining a taxane:AFP complex of the present invention, comprising (a) mixing AFP and a taxane in an aqueous vehicle, and (b) isolating AFP in a form complexed non-covalently with the taxane. In embodiments, the reaction product is filtered particularly through a polyethersulfone membrane filter. Most suitably, the filter is a 0.22 micron polyethersulfone membrane when pharmaceutical use is intended. In other embodiments the vehicle is an ethanol-supplemented (1-5% v/v) buffered saline vehicle.

These and other aspects of the present invention are now described in greater detail with reference to the accompanying figures in which:

### BRIEF DESCRIPTION OF THE DRAWINGS

FIG. 1 shows results from an in vivo study using AFP-Paclitaxel non-covalent complex (AFP-C-III-65- AOT1). (A) Survival data, (B) Cumulative tumor volume data, (C) Body weight of the mice in each group, and (D) Spleen weight of the mice in each group. Panels A and B clearly indicate the superiority of AFP-Paclitaxel complex over paclitaxel itself in its efficacy against the tumors established by COLO205 cell line in mice.

FIG. 2 provides a graph of survival data similar to that shown in FIG. 1 panel A, but using data from a subsequent study and with additional information concerning long term survival. There was also one more animal in the group represented here.

### DETAILED DESCRIPTION OF THE PREFERRED EMBODIMENTS

The present invention provides a pharmaceutically useful complex in which a highly hydrophobic cytotoxic drug such as one within the taxane family, and particularly paclitaxel, is bound non-covalently to α-fetoprotein, abbreviated AFP.

AFP is a human transporter protein produced in the fetus by embryonic liver and yolk sac. It enters the cells by endocytosis following binding to the specific AFP receptor. All embryonic cells express this receptor but receptor expression disappears once the embryo matures: human adult cells do not express the receptor, except in association with certain disease cells including certain cancer cells. The terms “alpha-fetoprotein”, “α-fetoprotein”, and “AFP” are used interchangeably herein with reference to the human protein having the 591-mer, mature sequence (residues 19-609) set out in UniProtKB designation P02771. Also useful in the present invention are those forms of AFP that bind to the AFP receptor, retain paclitaxel binding affinity, and occur as natural variants, such as the K187Q variant. As well, the present invention embraces the use of fragments of AFP that retain AFP receptor binding and paclitaxel binding affinity. The AFP protein includes at least three functional fragments that can be useful herein, which include the AFP 1 domain (residues 19-210 of the secretable precursor), AFP 2 domain (residues 211-402) and the AFP 3 domain (residues 403-601). Production of these fragments as recombinant products of E. coli expression are described for instance in Martinex's U.S. Pat. No. 6,416,734.

Furthermore, the present invention embraces the use of post-translationally modified forms of AFP, including those that incorporate more or less glycosylation than the natural human form thereof. In the human form, N-linked glycosylation occurs at Asn²⁵¹′ Thus, the natural form of human AFP is suitable for use in the present invention. Also useful are non-glycosylated forms of human AFP such as may be produced in prokaryotic host cells such as E. coli and Streptomyces. As well, alternatively glycosylated forms of human AFP are useful herein, such as those forms that are produced in eukaryotic hosts including yeast, Aspergillus, Pichia and the like, and in mammalian hosts that include CHO and COS cells. Also, the form of human AFP useful in the present invention includes human AFP produced in transgenic animals, including goats. Production of recombinant human AFP in transgenic animals generally, and in the milk of goats specifically, is described for instance in Merrimack's U.S. Pat. No. 7,208,576, which further describes the production of an unglycosylated form of AFP that incorporates a Asn²⁵¹ Gln substitution. In a preferred embodiment, the AFP is [Asn²⁵¹Gln]human AFP, as a non-glycosylated protein.

Thus, in embodiments of the present invention, the AFP used to prepare complexed taxane is recombinant human AFP, and includes the non-glycosylated human AFP mutant that is recoverable, for instance, from the milk of transgenic goats. As well, the recombinant AFP can be a non-glycosylated form produced by E. coli or by insect cells using the approach described in McGill's U.S. Pat. No. 6,331,611, where expression is driven from the trp and mal systems.

Alpha fetoprotein receptor (AFPR), as noted above, is typically associated with fetal tissue and is not present on adult cells, more than two months after birth. However, a large proportion of cancer cells express functional AFPR. Although this receptor has only been characterized partially, its existence has been unequivocally supported by experimental evidence. Moreover, monoclonal antibodies that bind this receptor are commercially available, and can be used to identify the protein, as well as cells that express the receptor. For example, the RDI division of Fitzgerald Industries in Concord Massachusetts provides two murine IgG2a monoclonal antibodies, designated 2B8 and 5E1, that bind the human AFPR. The same company also provides the receptor protein itself, as a reagent extracted from human fetal tissue. Identification of AFPR and cells that present it can also be achieved using a form of AFP that incorporates any detectable label. Tissue localization of the AFPR in mice has, for instance, recently been accomplished using an I¹²⁵-labeled form of human AFP, as described in Vestn Ross Akad Nauk, 2012, 4:11.

It will thus be appreciated that disease cells that can be targeted by the present complex are readily identified either by their immunoreactivity with AFPR antibodies, or by their binding affinity for AFP itself. These target cells are defined interchangeably herein either as being AFPR positive, or as having AFP-binding affinity.

Using the reagents just described, it will also be appreciated that different forms of AFP useful to deliver hydrophobic cytotoxic drugs, such as taxanes to a target disease cell can be identified by their ability to displace labeled AFP from binding with AFPR, or by their ability to engage AFPR directly. Cell-based assays are also useful for this purpose. In one example, AFPR-expressing U937 cells (a human male histiocytic lymphoma cell line available from ATCC under catalog number CRL 1593.2™) are exploited to confirm the AFPR binding affinity of any given form of AFP. The useful forms of AFP also will exhibit the required ability to form stable but non-covalent complexes with the selected taxane. That property is readily examined by mixing the selected form of AFP and the selected type of taxane under conditions disclosed herein. Suitable combinations will provide the taxane in a form complexed non-covalently with the AFP.

In one embodiment of the present invention, there are provided complexes formed by non-covalent interaction between AFP and the cytotoxic taxane, paclitaxel. As noted in the background section, paclitaxel is a very well-known cytotoxic product once extracted from the bark of the Pacific yew tree, and now produced either semi-synthetically or entirely synthetically, or by extraction from fungus associated with the arboreal source. Its structure is provided above. The present invention is applicable also to other taxanes that are analogs of paclitaxel that share its affinity for AFP binding and retain its desirable cytotoxicity. In one embodiment, the taxane is a paclitaxel analog known as docetaxel, and marketed as Taxotere®, having the structure shown below:

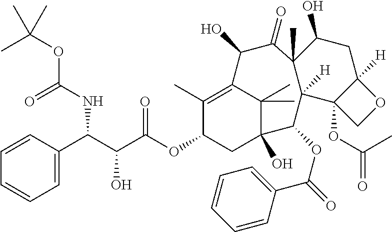

In another embodiment, the paclitaxel analog is a pharmaceutically acceptable salt form or ester of paclitaxel, including paclitaxel succinate. Another taxane that can be formulated in accordance with the present invention is Larotaxel, which is another semi-synthetic taxane currently in clinical development, having the structure (2α,3ξ,4α,5β,7α,10β,13α)-4,10-bis(acetyloxy)-13-({2R,3 S)-3-[(tert-butoxycarbonypamino]-2-hydroxy-3-phenylpropanoy}oxy)-1- hydroxy-9-oxo-5,20-epoxy-7,19-cyclotax-11-en-2-yl benzoate. The invention can equally be applied also to taxane-related compounds that are epothilones, such as Ixabepilone, and other compounds including cytotoxins that are poorly water soluble and require formulation in vehicles like Cremophor.

The invention is hereinafter described with reference specifically to paclitaxel, but is equally applicable to other AFP-binding taxanes.

Thus, in the present invention, the affinity of AFP for both paclitaxel and AFP receptor is exploited by producing AFP:paclitaxel complexes that are useful therapeutically to treat subjects presenting with disease cells that are AFP-binding, or AFPR positive. It has been found that AFP and paclitaxel bind with an affinity that is sufficient, during the course of complex preparation and following endogenous administration, that the paclitaxel is delivered selectively, and with reduced toxicity, by the associated AFP to the diseased cell. This approach to paclitaxel delivery could allow for reduced dosing of the cytotoxin and consequent reduction in associated adverse events, not only because the bound paclitaxel is less toxic but also because the cytotoxin is delivered selectively to AFPR positive diseased cells, thus sparing normal healthy cells. Moreover, the AFP:paclitaxel complex can be formulated in benign and standard pharmaceutical vehicles such as saline, thereby avoiding the use of carriers that in themselves create toxicity issues upon delivery to the patient.

To produce the AFP:paclitaxel complex, the AFP and paclitaxel are mixed in aqueous vehicle, desirably one that is isotonic, and has a pH that is physiological or mildly more acidic. In one embodiment, the mixing vehicle is phosphate buffered saline at a pH in the range from about 6 to about 7.5. In another embodiment, the mixing vehicle is water. In a further embodiment the mixing vehicle is saline (0.154M NaCl).

In a particular embodiment, the mixing vehicle is buffered saline with 1%-5% ethanol added. The ethanol can be substituted by a water soluble alcohol that is not disruptive to complex formation. For example and in the case where the alcohol is ethanol as a supplement, its presence in volume terms can be 1%, 2%, 3%, 4% and 5%, up to 10% (v/v).

Room temperature and standard pressure are acceptable mixing conditions. The mixing of the two reagents can be fostered using mild agitation, stirring, and the like. Formation of the paclitaxel:AFP complexes occurs during the mixing process, by a mechanism that is not entirely understood, although the binding is in the nature of an adsorption fostered by hydrogen bonding and/or hydrophobic interactions, and does not involve any chemical conjugation such as through covalent bonding or the like. AFP provides certain domains or “pockets” that are known to have an affinity for hydrophobic reagents, although other pockets that favour binding to amphiphilic or hydrophilic reagents are also presented by AFP. As a hydrophobic compound, paclitaxel likely adsorbs to AFP within its hydrophobic pockets while within the aqueous mixing environment.

After mixing, the complexed material can be separated from any insoluble, free material by standard techniques such as filtration to remove any unbound paclitaxel, to provide a preparation in which all of the AFP and paclitaxel is complexed, i.e., in a form essentially free from non-complexed paclitaxel. In a specific embodiment, the paclitaxel is added to water, and the AFP then is added until all of the paclitaxel dissolves, so that no insoluble material remains. Should any insoluble material remain, this can be filtered routinely, using for instance ultrafiltration against a 10K or 20K membrane.

In a preferred embodiment of the invention, the mixture is most suitably filtered using a membrane with low protein adsorption, such as a polyethersulfone membrane. This type of filter shows minimal taxane e.g., paclitaxel, retention relative to cellulose and ^{Teflon}®. Those membrane types were associated with separation of the complexes with about 50% of the taxane e.g., taxol retained thereon, and should be avoided.

The polyethersulfone (PES) membranes/filters are widely used in separation fields and particularly in biomedical fields. They are rigid and transparent within a broad temperature range. They are the reaction product of a diphenol and bis(4-chlorophenyl)sulfone, forming a polyether by elimination of sodium chloride. It has a low protein retention that makes it useful in biomedical applications including sterile drug filtration. In connection with AFP:taxane processing, it usefully permits retention of the complexes rather than their separation. Different commercial types of PES are available from numerous different suppliers.

The complex can thereafter be formulated immediately for therapeutic administration, stored briefly in its aqueous vehicle, or lyophilized for prolonged storage, as exemplified herein. Thus, in another embodiment, the present invention provides AFP:paclitaxel complex, in lyophilized form.

To produce a composition from which calculated unit dosages of paclitaxel can be prepared, the production of the complex desirably involves the use of predetermined amounts of each reagent. It has been found that one molecule of AFP (Molecular weight=66.5 kD) is able to complex, i.e., to bind and retain, up to about 4 molecules of paclitaxel (Molecular weight=854 kD), when mixing occurs under the conditions noted above. Thus, AFP is mixed desirably with an amount of paclitaxel sufficient to present up to about 4 moles (4±0.5 moles) of paclitaxel for each mole of AFP. The mixing can be controlled, of course, to produce AFP:paclitaxel complexes that comprise, on average, 3, 2, 1 or any fraction of a mole of paclitaxel for each mole of AFP. Although greater than 4 moles of paclitaxel can of course be mixed with each mole of AFP, it should be expected that excess paclitaxel will not dissolve, and will precipitate out of solution. Even should the excess paclitaxel bind to AFP, it will bind loosely and allow for the undesired release of free paclitaxel either in formulation or in vivo.

In one embodiment, the AFP:paclitaxel complex comprises about 4 moles of paclitaxel for each mole of AFP. In a related embodiment, the formulated or lyophilized complex is essentially free from uncomplexed paclitaxel. This allows for accurate unit dosage preparation and administration, calculated based on the amount of formulated paclitaxel.

It will be appreciated that a population of complexed AFP and paclitaxel molecules comprises complexes that are predominantly 4:1 (paclitaxel:AFP), as well as a relatively much smaller proportion of complexes having a different stoichiometry such as 3:1 and 5:1. The present disclosure thus qualifies these populations as being either “approximately” 4:1, or as being complexes that are 4:1 “on average”, to acknowledge the potential presence of a distribution within the complex population of a minor component of complexes that are not precisely 4:1 in stoichiometry.

For therapeutic use, the present invention provides AFP:paclitaxel complex as a pharmaceutical composition in which the complex is formulated with a pharmaceutically acceptable carrier. The formulation is adapted, in one embodiment, for intravenous administration, such as by injection or by infusion. Accordingly, the carrier desirably is an aqueous vehicle such as water for injection, saline, and the like.

The active ingredients to be used for in vivo administration will be sterile. This is readily accomplished by filtration through sterile filtration membranes.

Any other carriers, vehicles or excipients used in formulating the AFP-complexed paclitaxel must be chosen carefully, to avoid using agents or creating conditions that will disrupt the desired complex formation or will alter the binding affinity of AFP. Organic solvents should be avoided. Agents that introduce a pH outside physiological range, i.e., less than about pH 6 and more than about pH 8, should also be avoided for this reason. Water-soluble, inert carriers standard in the pharmaceutical formulation art are acceptable. They are, however, also unnecessary. The AFP:paclitaxel complex is readily formulated in water, or normal saline. Buffers are not required, and to the extent they alter tonicity adversely, they should also be avoided. The aqueous or saline solutions are ideal, in providing a physiologically tolerable pH and in being adapted for administration by the preferred routes of injection or infusion.

The AFP:paclitaxel complex is useful therapeutically in a similar manner and for treatment of the same indications as are already established for paclitaxel per se. Accordingly, and in one aspect, the present invention provides a method for treating a subject presenting with an AFPR positive, or AFP-binding, disease cell comprising administering to the subject an AFP:paclitaxel complex comprising AFP-bound paclitaxel in an amount effective to inhibit the growth and/or proliferation of that disease cell.

AFP receptor positive disease cells are readily identified both in vivo and ex vivo, using standard assays that employ detectable and selective AFP receptor binding ligands. Useful screens and assays are described hereinabove. AFPR positive disease cells that can be targeted by the present complexes include AFPR positive cancer cells, which include generally all cancer cells that bind AFP with specificity. Of course, it is anticipated that an effect may be seen only in those AFPR positive disease cells that respond to paclitaxel with the desired inhibition of growth or proliferation as reflected in reduced tumour size, or reduced tumour growth rate. Such cells and tumours have the character of being “taxane-responsive, e.g., “paclitaxel-responsive”, and are the preferred targets for treatment with the present complexes. In addition, certain paclitaxel-resistant cancer cells in which the resistance to paclitaxel is due to overexpression of membrane pumps that actively remove paclitaxel from the cells could be effectively treated with AFP:paclitaxel formulation.

Any appropriate route of administration can be employed, for example, parenteral, intravenous, intramuscular, intracranial, intraorbital, intraventricular, intracapsular, intraspinal, intracisternal, and intraperitoneal administration. Administration by injection or infusion is preferred.

For the treatment of subjects presenting with cancer cells that bind AFP, the appropriate dosage of an AFP:paclitaxel complex will depend on the type of disease to be treated, as defined above, the severity and course of the disease, whether the agent is administered for therapeutic purposes, previous therapy, the patients clinical history and response to the agent, and the discretion of the attending physician. The agent is suitably administered to the patient at one time or over a series of treatments. Progression of disease can be monitored in accordance with practice standard in paclitaxel therapy.

For example, depending on the type and severity of the disease, about 100 μg/kg to 2 mg/kg of paclitaxel, present as AFP complex, is a candidate dosage for administration to the patient, whether, for example, by one or more separate administrations, or by infusion. For repeated administrations over several days or longer, depending on the condition, the treatment is sustained until a desired suppression of disease symptoms occurs or until progression of the disease is observed. However, other dosage regimens may be useful. Unit doses based on the weight of paclitaxel in the complex can be in the range, for instance of about 500 ug to 500 mg, such as 1 mg, 5 mg, 10 mg, 25 mg, 50 mg, 100 mg, 150 mg, 200 mg, 250 mg and 300 mg. Unit doses based on the weight of AFP-complexed paclitaxel will have a weight that is about 20 times that of an equivalent unit dose of non-complexed paclitaxel, e.g. will lie in the range from about 10 mg to about 5,000 mg. For instance, a dose of 2,000 mg AFP:paclitaxel will deliver about 100 mg of paclitaxel to the subject, which is within the range of clinical doses used in a weekly dosing regimen. Of course, the formulated complex can be provided in multidose form, comprising 2, 3, 4, 5 or more unit doses within each container, e.g., vial. The complexed preparation can also be provided in kit form, comprising a lyophilized preparation comprising the complex and a separately packaged vehicle for reconstitution of the preparation into an administrable dosage form. In the alternative, the kit may simply comprise the complexed preparation, and instructions for the reconstitution thereof into an administrable dosage form. The progress of anti-cancer therapy is monitored by techniques and assays established for the particular disease being treated.

For non-complexed paclitaxel, standard dosing has been established as follows:

For the treatment of ovarian carcinoma, as first line therapy, paclitaxel 175 mg/m2 infused over 3 hours followed by cisplatin 75 mg over 6 courses; as second line therapy, paclitaxel 135-170 mg/m2 infused over 24 hours by continuous infusion;

For the treatment of breast carcinoma, as adjuvant therapy, paclitaxel 175 mg/m2 as a 3 hour infusion every 3 weeks for 4 courses, with doxorubicin and/or cyclophosphamide; and

For the treatment of AIDS-related Kaposi's sarcoma, paclitaxel 135 mg/m2 as a 3 hour infusion every 3 weeks, with dose escalation to 155 mg/m2 and 175 mg/m2 as permitted.

New regimens using weekly administration of paclitaxel 60 to 80 mg/m2 have been shown to improve efficacy and are now being used clinically by many physicians.

In embodiments of the present invention, the administered dose of paclitaxel, in AFP-complexed form, is from 25% to 100%, e.g., about 25%, 40%, 50%, 60%, 70% or 75%, of the unit dose employed for non-complexed paclitaxel.

As well, where the toxicity of non-complexed paclitaxel requires infusion at relatively slow rates, it is anticipated that the present complexed paclitaxel can be administered at an accelerated rate, such as an infusion over 5 or 10 minutes.

It will thus be appreciated that an effective amount of the complex is an amount effective as a unit dose or as part of a treatment regimen that retards or inhibits the growth or proliferation of disease cells that are paclitaxel-responsive and positive for AFP binding.

The paclitaxel complexes are useful in the treatment of a variety of paclitaxel-responsive cancers, to inhibit the growth or proliferation of cancer cells and tumours comprising them, including hematopoietic cell cancers and solid tumours. Conditions or disorders to be treated include malignant tumors (e.g., renal, liver, kidney, bladder, breast, gastric, ovarian, colorectal, prostate, pancreatic, lung, vulva, and thyroid); hepatic carcinomas; sarcomas; glioblastomas; and various head and neck tumors; leukemias and lymphoid malignancies.

It will be appreciated that the subjects treated with the present complexes should be at least about 3 months old so that endogenous AFP receptor is not prevalent on the subject's healthy cells and tissue.

The complex can be administered to a subject in need thereof in combination with useful other agents. Administration “in combination with” one or more further therapeutic agents includes simultaneous (concurrent) and consecutive administration in any order. Other therapeutic regimens may be combined with the administration of the anti-cancer agent of the instant invention. For example, the patient to be treated with such anti-cancer agents may also receive radiation therapy, such as external beam radiation. Alternatively, or in addition, a chemotherapeutic or biologic agent may be administered to the patient. Preparation and dosing schedules for such chemotherapeutic or biologic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992). The chemotherapeutic agent may precede, or follow administration or the complex, or may be given simultaneously therewith. The complex may be combined with any other drug particularly including irinotecan (CPT-11), cisplatin, cyclophosphamide, melphalan, dacarbazine, doxorubicin, pemetrexed, daunorubicin, and topotecan, Herceptin®, as well as tyrosine kinase inhibitors and the like.

In another embodiment of the invention, an article of manufacture containing AFP:paclitaxel complex useful for the treatment of the disorders described herein is provided. The article of manufacture comprises the present complex, optionally and suitably in lyophilized form, in a container and suitably bearing a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or vial having a stopper pierceable by a hypodermic injection needle). The label on or associated with, the container indicates that the composition is used for treating a cancer condition. The article of manufacture may further compromise a second container comprising a pharmaceutically-acceptable buffer, such as phosphate-buffered saline, normal saline, water for injection, and the like. It may further include other matters desirable from a commercial and use standpoint, including other buffers, diluents, filters, needles, syringes, and package inserts with instructions for use in accordance with the present invention. Control agents or standards useful in the method can also be included in the kit, such as an AFP preparation standard.

### EXAMPLES

A clinically used cytotoxic drug, paclitaxel or taxol was non-covalently complexed with human recombinant AFP (the non-glycosylated [Asn²⁵¹Gln]human AFP form) to generate an AFP:paclitaxel non-covalent complex that is stable in this complexed form. Further, it is determined that a range of 3-5 paclitaxel molecules, optimally 4 molecules of paclitaxel, would bind approximately to one molecule of AFP non-covalently, and can still maintain the integrity of the protein structure of AFP. Such a complex has many fold higher effective molar solubility for paclitaxel than for paclitaxel itself without the presence of AFP, in aqueous solutions such as saline. As well, it is demonstrated that the complex exhibits equal or better potency in the in vitro antiproliferative assays with the cell lines carrying AFP receptor, due to the AFP receptor-mediated uptake of the paclitaxel complex and the release of the drug within the cancer cell. Moreover, the complex will exhibit superior efficacy in the cancer mouse models where the AFPR receptor is expressed on the cancer cells, in comparison to paclitaxel itself, and reduced systemic toxicity to the animals. Finally, the effective amount of paclitaxel that would be required in the complexed form is less than required for paclitaxel alone in the treatment of cancer.

General Description for the Preparation of AFP Non-covalent Complex with Taxanes.

Various formulations comprising paclitaxel: AFP complex were prepared for testing, as described below:

The general preparation procedure for the paclitaxel-AFP complex developed via various trials described here resulted in a soluble and stable complex when paclitaxel:AFP molar ratio is approximately 4:1. Although greater than 4 molar equivalents of paclitaxel can of course be mixed with AFP, it was found that excess paclitaxel either would not dissolve to create a stable homogeneous solution, or may precipitate out of aqueous solution upon standing. Below, a number of preparations of paclitaxel:AFP complex are described.

Preparation of Paclitaxel-AFP Non-covalent complex (Alpha-C-III-64-AOT).

Paclitaxel (8.7 mg) was suspended in 90.0 mL of saline solution (pH 6.5) and sonicated for 20 min. AFP (8.342, 20 mg/mL) was added drop wise with gentle shaking at r.t. in a period of 30 min. Saline solution (3.538 mL) was added to take the sample to a final volume of 101.88 mL. The sample was left shaking (Innova-42 incubator- shaker) for 1 hour at r.t., labeled and kept at 4° C. (molar ratio Paclitaxel:AFP is 4:1, 100 uM Paclitaxel).

Preparation of Paclitaxel-AFP Non-covalent complex (Alpha-C-III-65-AOT1).

Paclitaxel (6.5 mg) was suspended in 65.0 mL of deionized water (pH 6.5) and sonicated for 20 min. AFP (6.232 mL, 20 mg/mL) was added drop wise with gentle shaking at r.t. in a period of 30 min. Deionized water (4.888 mL) was added to take the sample to a final volume of 76.12 mL. The sample was left shaking (Innova-42 incubator-shaker) for 1 hour at r.t., labeled and kept at 4° C. (molar ratio Paclitaxel:AFP is 4:1, 100 uM Paclitaxel, 25 uM AFP, concentration labeled as 1× concentration pre-lyophilization). From this conjugate the following samples were prepared:

Paclitaxel-AFP Non-covalent complex (Alpha-C-III-65-AOT5).

75.8 mL of sample Alpha-C-III-65 was transferred to a 300 mL beaker, frozen at −80oC and lyophilized for 48 h. The final product (white powder), was reconstituted in 12.636 mL of saline solution (pH 6.5), to yield a turbid white solution (molar ratio Paclitaxel:AFP is 4:1, 600 μM Paclitaxel, 150 uM AFP, concentration of paclitaxel 6x the pre- lyophilized product).

Paclitaxel-AFP Non-covalent Complex (Alpha-C-III-65-AOT4).

Alpha-C-III-65-AOT5 (4 mL) was diluted with saline solution (2 mL, pH 6.5) to a final volume of 6 mL. (molar ratio Paclitaxel:AFP is 4:1, 400 uM Paclitaxel, 100 μM AFP, concentration of paclitaxel is 4× the pre-lyophilized product).

Paclitaxel-AFP Non-covalent Complex (Alpha-C-III-65-AOT3).

Alpha-C-III-65-AOT5 (2 mL) was diluted with saline solution 4 mL, pH 6.5) to a final volume of 6 mL. (molar ratio Paclitaxel:AFP is 4:1, 200 uM Paclitaxel, 50 uM AFP, concentration of paclitaxel 2× the pre-lyophilized product).

Paclitaxel-AFP Non-covalent Complex (Alpha-C-III-65-AOT2).

Alpha-C-III-65-AOT5 (1 mL) was diluted with saline solution (5 mL, pH 6.5) to a final volume of 6 mL. (molar ratio Paclitaxel:AFP is 4:1, 100 uM Paclitaxel, 25 uM AFP, concentration of paclitaxel is same as that of pre-lyophilized product).

NOTE: Alpha-C-III-65-AOT1 is a pre-lyophilized product. Alpha-C-III-65-AOT2, Alpha-C-III-65-AOT3, Alpha-C-III-65-AOT4, and Alpha-C-III-65-AOT5 are post-lyophilized samples.

Preparation of Paclitaxel-AFP Non-covalent Complex (Alpha-C-III-68-AOT1).

Paclitaxel (17.8 mg) was suspended in 185.0 mL of deionized water (pH 6.5) and sonicated for 30 min. AFP (17.066 mL, 20 mg/mL) was added drop wise with gentle shaking at r.t. in a period of 30 min. Deionized water (6.384 mL) was added to take the sample to a final volume of 208.45 mL. The sample was left shaking (Innova-42 incubator-shaker) for 1 hour at r.t., labeled and kept at 4 ° C. (molar ratio Paclitaxel:AFP is 4:1, 100 uM)

Paclitaxel, 25 uM AFP, concentration Ix pre-lyophilization). From this complex, Alpha-C-III-68-AOT1, the following samples were prepared:

Paclitaxel-AFP Non-covalent Complex (Alpha-C-III-68-AOT2).

12 mL of Alpha-C-III-68 were transferred into a 20 mL vial, frozen at −80 ° C. and lyophilized for 48 h (16 samples were prepared in parallel and lyophilized). The final product (white powder), was labeled and kept at 4 ° C. The samples should be reconstituted in 2 mL of saline solution (pH 6.5) before use (molar ratio Paclitaxel:AFP is 4:1, 600 ₁AM Paclitaxel, 150 μM AFP, concentration of paclitaxel is 6x pre-lyophilized product).

A circular dichroism experiment indicated that there is no change in the folding of AFP, after it is complexed with paclitaxel, in the pre-lyophilized complex, and in the reconstituted saline solution post-lyophilization.

Anti-cancer Activities

| | | | IC₅₀ ± std. |
|---|---|---|---|
| | | IC₅₀ ± std. error | error (nM) |
| | Sample | (nM) for Pac | calculated |
| Compound code | description | or Pac-succinate | for AFP |
| Paclitaxel | Paclitaxel in PBS | 3.4 ± 0.8 | — |
| | pH 7.4 | | |
| α-C-III-41-AOT | AFP-Paclitaxel | — | 0.6 ± 0.3 |
| | equilibrium in PBS | | |
| | pH 7.4 | | |
| | 10X, 1 wash | | |
| α-C-III-48-AOT | AFP-Paclitaxel in | 1.6 ± 2.0 | 0.5 ± 0.6 |
| | PBS pH 7.4 | | (based on |
| | Non-covalent, | | loading |
| | equilibrium | | efficiency) |
| | titration | | |
| Paclitaxel- | Paclitaxel- | 26.5 ± 11.3 | — |
| Succinate | succinate in PBS | | |
| | pH 7.4 | | |
| α-C-III-56-AOT | AFP-Paclitaxel- | 11.6 ± 4.3 | 2.5 ± 0.8 |
| | succinate in PBS | | (based on |
| | pH 7.4 | | loading |
| | Non-covalent, 5X, | | efficiency) |
| | no wash | | |

In vitro activities of Alpha-C-III-65-* Samples: There is a difference in the in vivo cytotoxic potency of these samples.

| Sample | IC₅₀ (nM) COLO-205 cells |
|---|---|
| Alpha-C-III-65 AOT1 | 16.4 ± 2.1 |
| Alpha-C-III-65 AOT2 | 3.9 ± 1.7 |
| Alpha-C-III-65 AOT3 | 9.0 ± 1.2 |
| Alpha-C-III-65 AOT4 | 5.0 ± 0.8 |
| Alpha-C-III-65 AOT5 | 3.0 ± 1.6 |

In Vivo Studies.

The following protocol was used for the in vivo studies in mice. Mice were injected sub-cutaneously in the right flank with 1.5×10⁶ COL0225. Treatment began one week later.

| | # of mice per group | |
|---|---|---|
| | 4 week old | 9 week old |
| untreated (no cells, no treatment) | 1 | 1 |
| PBS injected IV 3 times per week for | 5 | 1 |
| 4 weeks | | |
| Paclitaxel AFP 2.6 mg/kg injected IV/IP | 5 | 1 |
| 5 times per week for 4 weeks | | |
| Paclitaxel 2.6 mg/kg injected IV/IP | 5 | 1 |
| 5 times per week for 4 weeks | | |
| Total | 16 | 4 |

Results from In Vivo Experiments.

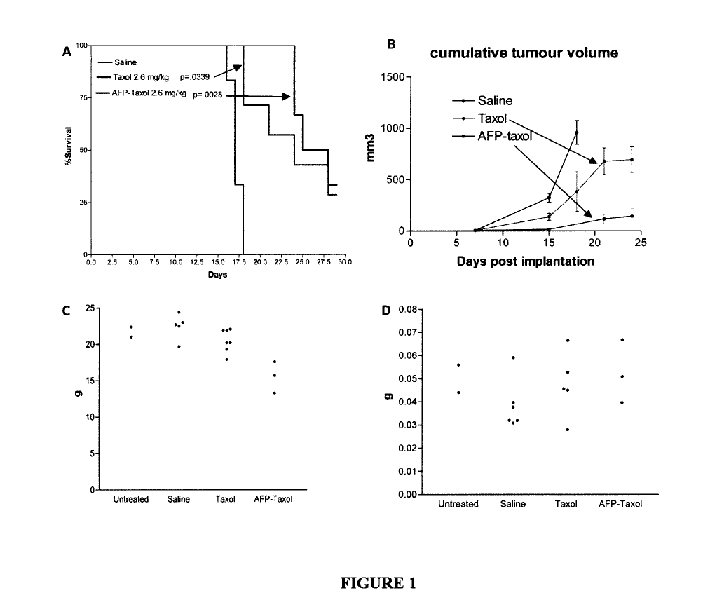

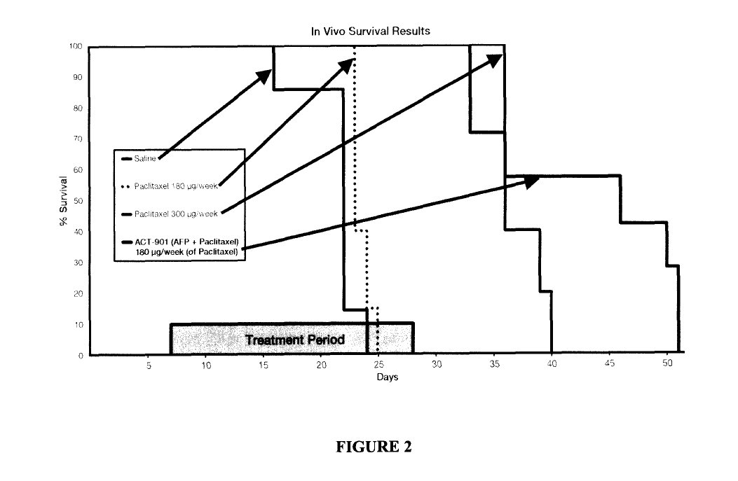

FIG. 1A and FIG. 2 show the general survival data from the in vivo experiment conducted using AFP-Paclitaxel non-covalent complex. General efficacy and survival rate in the group treated with AFP- Paclitaxel complex is higher than that treated with Paclitaxel alone and that receiving only saline.

Profiles on (C) body weights, (D) spleen weights, and (B) tumor volumes in mice from each group are also shown. Measures of cumulative tumor volume clearly demonstrate the superior efficacy of AFP- Paclitaxel complex in comparison to Paclitaxel itself, and complements the survival graph.

Although preferred embodiments of the invention have been described herein, it will be understood by those skilled in the art that variations may be made thereto without departing from the spirit of the invention or the scope of the appended claims. All documents disclosed herein are incorporated by reference.

We claim:

1. A complex comprising a taxane and α-fetoprotein (AFP), wherein the AFP and taxane are complexed non-covalently and the taxane is present, on average, at up to about 4 molecules per molecule of AFP.

2. The complex according to claim 1, wherein the paclitaxel is present, on average, at 4 molecules of paclitaxel per molecule of AFP.

3. The complex according to claim 1 or claim 2, wherein the AFP is recombinant human AFP.

4. The complex according to claims 1-3, wherein the AFP has the primary amino acid sequence of human AFP.

5. The complex according to claims 1-3, wherein the AFP has the primary amino acid sequence of [Asn²⁵¹Gln]human AFP.

6. The complex according to claims 1-5, wherein the AFP is a non-glycosylated AFP.

7. The complex according to claim 6, wherein the non-glycosylated AFP is produced by transgenic goats.

8. The complex according to claims 1-7, wherein the taxane is paclitaxel.

9. The complex according to claims 1-7, wherein the taxane is docetaxel.

10. The complex according to claims 1-9, in lyophilized form.

11. A preparation comprising a complex according to claims 1-10, wherein the preparation is essentially free from non-complexed taxane.

12. A pharmaceutical composition, comprising the complex of claims 1-9, and a pharmaceutically acceptable carrier.

13. The pharmaceutical composition according to claim 12, wherein said carrier is an aqueous vehicle.

14. The pharmaceutical composition according to claim 13, wherein the aqueous vehicle is saline.

15. The pharmaceutical composition according to claim 12, wherein the taxane is present in a unit dose effective, in a treatment regimen, to inhibit the growth or proliferation of a cell that is AFP receptor positive.

16. The pharmaceutical composition according to claims 12-14, wherein the taxane is paclitaxel.

17. A method for inhibiting the growth or proliferation of a disease cell that is AFP receptor positive and taxane-responsive, comprising treating the cell with an effective amount of a complex according to claims 1-9.

18. A method for treating a subject presenting with an AFP receptor positive and taxane-responsive disease cell, comprising administering to the subject an amount of the complex of claims 1-9 effective to inhibit the growth or proliferation of said cell.

19. A method for obtaining a complex according to claims 1-9, comprising:
(a) mixing AFP and a taxane in an aqueous vehicle, and
(b) isolating AFP in a form complexed non-covalently with the taxane.

20. The method according to claim 19, wherein the step of mixing AFP and taxane is performed by adding AFP to an aqueous suspension comprising said taxane.

21. The method according to claims 20 and 21, wherein the AFP and paclitaxel are present, on average, at a ratio of 1 mole of AFP for every 4 moles of taxane.

22. The method according to claims 19-21, wherein the taxane is paclitaxel.

23. The method according to claims 19-22, wherein the AFP is non-glycosylated [Asn²⁵¹Gln]human AFP.

24. The method according to claims 19-23, comprising the further step of combining the complex with a pharmaceutically acceptable carrier.

25. The method according to claims 19-24, wherein the step of filtering the product resulting from the mixing of taxane and AFP is conducted using a polyethersulfone membrane.

26. The method according to claim 25, wherein the membrane is a 0.22 micron filter.

27. The method according to claims 19-26 wherein the aqueous vehicle within which the AFP and taxane are mixed is an ethanol-supplemented buffered saline solution.

28. The method according to claim 27 wherein the aqueous vehicle is supplemented with 1% to 5% ethanol (v/v).

29. The use of a complex according to any of claims 1-9 in the preparation of a medicament for the treatment of a taxane-responsive, AFP receptor positive disease cell.

30. A pharmaceutical composition for use in treating a taxane-responsive disease cell that is AFP receptor positive, comprising a complex according to claims 1-9 and a pharmaceutically acceptable carrier.

31. A method for preparing paclitaxel for use in treating cancer, comprising the step of mixing a taxane with AFP to cause formation of a complex in which AFP and taxane are bound non-covalently, and filtering the result thereof through a polyethersulfone membrane.

32. A kit comprising a complex according to any one of claims 1-10, and instructions for reconstitution thereof into an injectable or infusible formulation for the treatment of AFP receptor positive, taxane-responsive cancer.

33. The kit according to claim 32, wherein the complex is in lyophilized form.
